(19) United States
(12) Patent Application Publication
(10) Pub. No.: US 2021/0183480 A1
(43) Pub. Date: Jun. 17, 2021

(54) Biometric Authentication for Access to Medical Information on a Distributed Ledger

(71) Applicant: AT&T Intellectual Property I, L.P., Atlanta, GA (US)

(72) Inventors: Umayal Roja Swarnam, Aurora, IL (US); Mike Iapalucci, Carrollton, TX (US); Lars Johnson, Brooklyn, NY (US)

(73) Assignee: AT&T Intellectual Property I, L.P., Atlanta, GA (US)

(21) Appl. No.: 16/714,517

(22) Filed: Dec. 13, 2019

(51) Int. Cl. G16H 10/60; G06F 16/248; G06F 16/23; G06F 21/32

(52) U.S. Cl. CPC G16H 10/60; G06F 16/248; H04L 9/3268; G06F 21/32; G06F 16/2379

(57) ABSTRACT

A permissioned blockchain network can include a plurality of peer nodes and an orderer node. A first peer node of the plurality of peer nodes can be associated with a plurality of patients. A second peer node of the plurality of peer nodes can be associated with a plurality of medical professionals. A third peer node of the plurality of peer nodes can be associated with a plurality of caregivers. A plurality of channels can be used by the plurality of peer nodes to communicate and to update a plurality of ledgers. A first ledger of the plurality of ledgers can include medical history data associated with the plurality of patients. A second ledger of the plurality of ledgers can include prescription history data associated with the plurality of patients. A third ledger of plurality of ledgers can include remote patient monitoring device data associated with the plurality of patients.

### BACKGROUND

Blockchain is emerging as a preeminent distributed ledger and is receiving increasing attention from researchers, practitioners, organizations, and the public. Initially, blockchain was developed to address the “double spending” problem in cryptocurrencies, but recently, many new applications of blockchain have been proposed or are being developed. Blockchain allows sharing data in a decentralized, transparent and immutable way, using a peer-to-peer network, without the need to trust any particular entity.

Blockchain is receiving growing attention not just as the underlying technology of cryptocurrencies, but also as a public ledger in various domains. Financial institutions, for example, are examining the use of blockchain as a ledger for financial transactions to cut out the middleman to reduce costs and to expedite processing transactions. Blockchain also can be used to maintain digital assets, such as stocks, bonds, land titles, and the like. Stored transactions record the transfer of assets between users. Blockchain can store data and documents, either in full or merely a digest of the data (e.g., a cryptographic hash like SHA-256), to provide evidence of the existence of data or documents, such as contracts, patents, scientific publications, deeds, insurance policies, and the like. Blockchain also can be used for identity management through hashed features of a person (e.g., verifiable attributes of the person) stored with a public key or some other means to electronically sign documents, or access remote services to protect people from identity theft and fraudulent impersonation. Blockchain has the potential to provide a secure infrastructure for smart cities and could facilitate the creation of a marketplace of social data where people share their private data for public benefit. Blockchain also has commercial uses, such as for tracking diamonds from mines to market, managing data provenance in Internet of Things (“IoT”) systems, providing transparency in product manufacturing and supply chain management, and supporting vehicle provenance.

Health data is considered one of the most sensitive types of data about an individual, and under the Health Insurance Portability and Accountability Act (“HIPAA”) in the United States, must be protected with confidentiality. While hospitals and other medical facilities may use walled gardens to control access to patient health data, a more recent approach is to allow health care professionals (e.g., doctors, nurses, radiography technicians, etc.) access to patient health data that has been pseudo-anonymized. After patient health data are anonymized and can no longer be linked to a specific patient, the patient health data may be rendered useless to a malicious attacker. However, access to patient health data is crucial in emergency situations, and the anonymity of patient health data may prove troublesome if the patient is unable to provide identification when they require immediate treatment. Certain health data may be vital for first responders or for doctors to provide an immediate, accurate diagnosis and proper treatment. While the need for accurate health data for care is clear, threats from identity theft and lawsuits have made it critical to protect patient's personal information throughout the diagnostic and treatment processes.

### SUMMARY

Concepts and technologies disclosed herein are directed to biometric authentication for access to medical information on a distributed ledger. According to one aspect disclosed herein, a biometric authentication device can be used to identify a patient. The biometric authentication device can include one or more biometric sensors. The patient can be identified, at least in part, via biometric data obtained by the biometric sensor(s). A device can execute a blockchain application to connect the device to a peer node. In some embodiment, the device is or includes the biometric authentication device. The peer node can host a chaincode and a ledger. The ledger can store health data associated with the patient. The device can generate a proposal directed to the peer node. The proposal can be used to invoke the chaincode to interact with the ledger with regard to the health data. For example, the proposal may be to query the ledger for at least a portion of the health data, or to update the ledger such as to add, remove, and/or modify at least a portion of the health data. The device can send the proposal to the peer node. The device can receive a proposal response from the peer node. The proposal response can include a query result that includes the portion of the health data identified in the proposal. Alternatively, the proposal response can include a proposed ledger update.

In some embodiments, the ledger can be associated with a specific channel of a plurality of channels associated with the health data. A plurality of peer nodes, including the peer node, and the blockchain application can be associated with the specific channel. The ledger can include different types of health data. For example, the ledger can include medical history data associated with the patient, patient prescription history data associated with the patient, or remote patient monitoring device data associated with the patient.

In some embodiments, in response to the device receiving the proposed ledger update, the device can send the proposed ledger update to a plurality of endorser peer nodes. The device can receive, from the plurality of endorser peer nodes, endorsements of the proposed ledger update. If the device receives an endorsements from each of the plurality of endorser peer nodes, the device can determine that consensus among the plurality of endorser peer nodes has been achieved, and the proposed ledger update should be realized. If consensus is achieved, the device can generate and send a transaction order request to an orderer node. The orderer node can generate one or more transaction blocks based upon the transaction order request. The transaction blocks can be used to update the ledger in accordance with the proposed ledger update. The device can receive a ledger update event to indicate that the ledger has been updated by the peer node in accordance with the proposed ledger.

According to another aspect disclosed herein, a permissioned blockchain network can include at least one organization. The organization can include a plurality of peer nodes and an orderer node. A first peer node of the plurality of peer nodes can be associated with a first group of users such as a plurality of patients. A second peer node of the plurality of peer nodes can be associated with a second group of users. The second group of users can include a plurality of medical professionals. A third peer node of the plurality of peer nodes can be associated with a third group of users. The third group of users can include a plurality of caregivers.

The permissioned blockchain network also can include a plurality of channels through which the plurality of peer nodes communicate to update a plurality of ledgers. Each ledger of the plurality of ledgers can be associated with one channel of the plurality of channels. For example, a first ledger of the plurality of ledgers can include medical history data associated with the plurality of patients. A second ledger of the plurality of ledgers can include prescription history data associated with the plurality of patients. A third ledger of plurality of ledgers can include remote patient monitoring device data associated with the plurality of patients.

The permissioned blockchain network can include a device. The device can include a processor that executes a blockchain application to perform operations. In particular, the blockchain application can connect the device to a specific peer node of the plurality of peer nodes. The blockchain application can generate a proposal directed to the specific peer node. The proposal can be used to invoke a specific chaincode to interact with a specific ledger. The blockchain application can send the proposal to the specific peer node. The blockchain application can receive a proposal response from the specific peer node.

The permissioned blockchain network can include a biometric authentication device. The biometric authentication device can be used to identify a specific patient of the plurality of patients via biometric data obtained from a biometric sensor of the biometric authentication device. The biometric authentication device can be part of the device that executes the blockchain application or a standalone device.

In some embodiments, the proposal is to query the specific ledger for at least a portion of health data stored by the specific ledger. The health data can include the medical history data associated with the specific patient, the prescription history data associated with the specific patient, or the remote patient monitoring device data associated with the specific patient. In these embodiments, the proposal response can include a query result that includes at least the portion of the health data.

In some embodiments, the proposal is to update the specific ledger. For example, the proposal may be to add, remove, or modify at least the portion of the health data. In these embodiments, the device can execute a blockchain application to receive the proposal response that includes a proposed ledger update. The blockchain application can send the proposed ledger update to a plurality of endorser peer nodes. The blockchain application can receive, from the plurality of endorser peer nodes, endorsements of the proposed ledger update such that consensus among the plurality of endorser peer nodes is achieved. The blockchain application can generate and send a transaction order request to the orderer node. The orderer node generates a transaction block based upon the transaction order request to update the specific ledger in accordance with the proposed ledger update. The blockchain application can receive a ledger update event to indicate that the specific ledger has been updated by the specific peer node in accordance with the proposed ledger update.

It should be appreciated that the above-described subject matter may be implemented as a computer-controlled apparatus, a computer process, a computing system, or as an article of manufacture such as a computer-readable storage medium. These and various other features will be apparent from a reading of the following Detailed Description and a review of the associated drawings.

This Summary is provided to introduce a selection of concepts in a simplified form that are further described below in the Detailed Description. This Summary is not intended to identify key features or essential features of the claimed subject matter, nor is it intended that this Summary be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIGS. 1A-1C are block diagrams illustrating aspects of a permissioned blockchain network capable of implementing aspects of the concepts and technologies disclosed herein.

FIGS. 2A-2B is a flow diagram illustrating aspects of a method for querying or updating a ledger that contains patient data, according to an illustrative embodiment.

FIG. 3 is a block diagram illustrating an example computer system and components thereof capable of implementing aspects of the embodiments presented herein.

FIG. 4 is a block diagram illustrating an example mobile device and components thereof capable of implementing aspects of the embodiments presented herein.

FIG. 5 is a block diagram illustrating an example network capable of implementing aspects of the embodiments presented herein.

### DETAILED DESCRIPTION

The concepts and technologies disclosed herein allow for a subset of a patient's data to be provided to those with a need to provide care to the patient in an emergency or other medical situation. The concepts and technologies disclosed herein utilize a distributed ledger-based system that provides a limited trust environment in contrast to the existing fragmented system that entrusts sensitive data with third parties like medical professionals, patient caregivers, insurance companies, pharmacies, and other health-related entities. The distributed ledger-based system can prevent a single point of failure. This is particularly important for healthcare applications due to the time and precise requirements of health data. Additionally, the distributed ledger-based system can allow users to exchange data between various organizations, while retaining an audit log to track who has given access, received access, and what data was accessed by each user.

The concepts and technologies disclosed herein also implement biometrics to augment the authentication for a third party user to access some data from a distributed ledger without accessing personally identifiable information like name, address, social security number, and the like. A biometric device can provide a limited, temporary authentication to access some data, while the distributed ledger can provide the proper authorization for the user who requests access to the distributed ledger.

While the subject matter described herein may be presented, at times, in the general context of program modules that execute in conjunction with the execution of an operating system and application programs on a computer system, those skilled in the art will recognize that other implementations may be performed in combination with other types of program modules. Generally, program modules include routines, programs, components, data structures, computer-executable instructions, and/or other types of structures that perform particular tasks or implement particular abstract data types. Moreover, those skilled in the art will appreciate that the subject matter described herein may be practiced with other computer systems, including hand-held devices, mobile devices, wireless devices, multiprocessor systems, distributed computing systems, microprocessor-based or programmable consumer electronics, minicomputers, mainframe computers, routers, switches, other computing devices described herein, and the like.

In the following detailed description, references are made to the accompanying drawings that form a part hereof, and in which are shown by way of illustration specific embodiments or examples. Referring now to the drawings, in which like numerals represent like elements throughout the several figures, aspects of concepts and technologies for biometric authentication for access to medical information on a distributed ledger will be described.

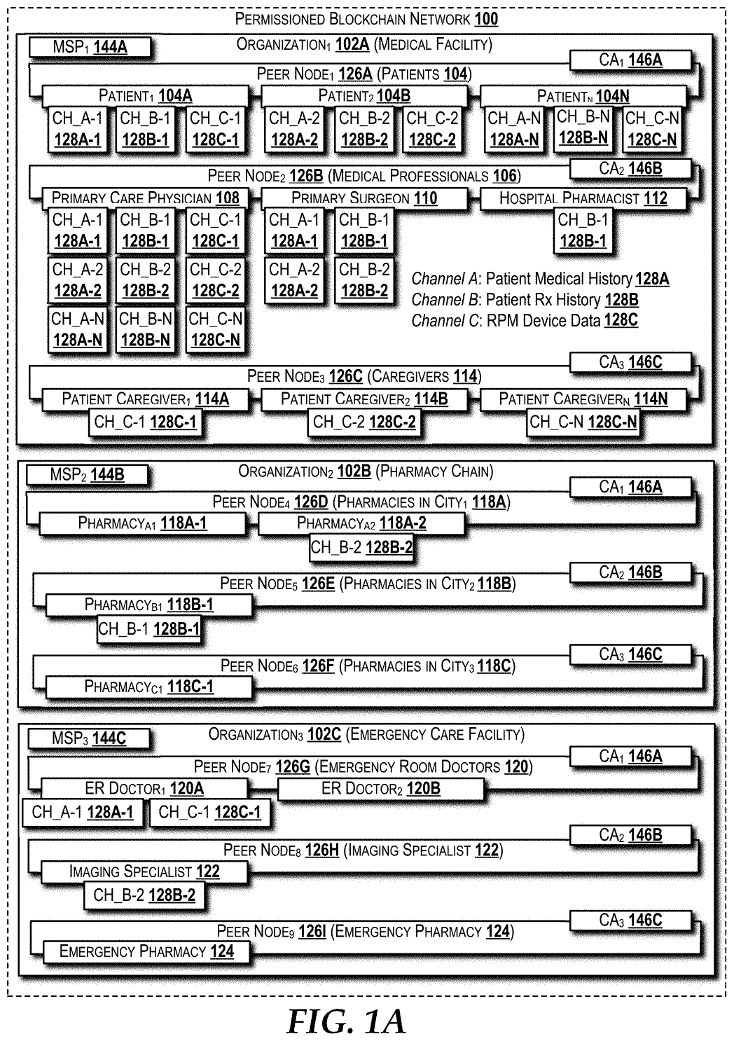

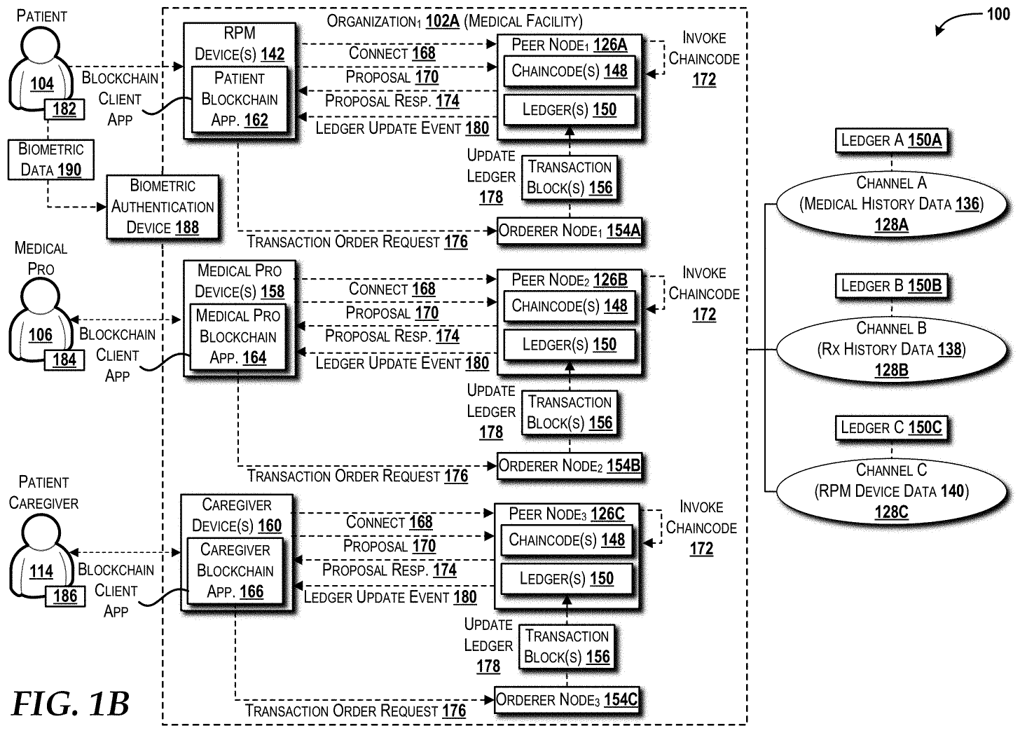

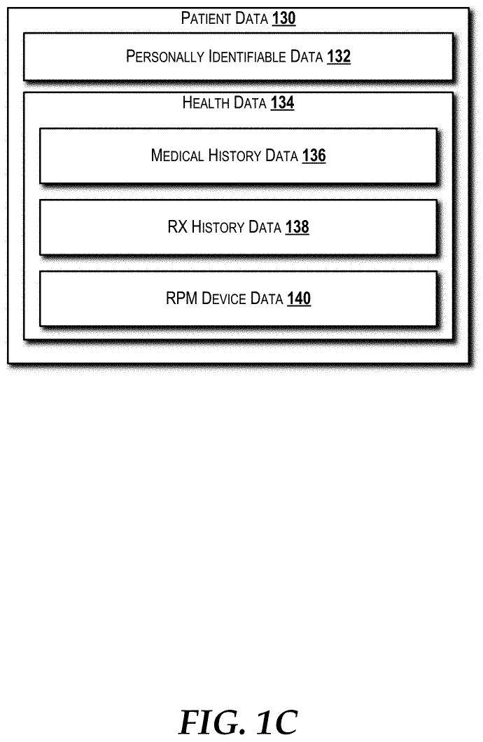

Turning now to FIG. 1A, a permissioned blockchain network 100 capable of implementing aspects of the concepts and technologies disclosed herein will be described, according to illustrative embodiment. The permissioned blockchain network 100 will be described herein in context of Hyperledger® (available from The Linux Foundation®), and for this reason, certain terminology used herein will be consistent with Hyperledger® specifications. It should be understood, however, that the use of this terminology is not intended to limit the scope of the accompanying claims to any particular permissioned blockchain platform or technology. Those skilled in the art will appreciate the applicability of the concepts and technologies disclosed herein to any permissioned blockchain platform or technology. Moreover, blockchain technology is described herein as a particular type of distributed ledger technology, although other distributed ledger technologies are contemplated.

The illustrated permissioned blockchain network 100 includes a first organization (“organization₁”) 102A, a second organization (“organization”) 102B, and a third organization 102C, although the permissioned blockchain network 100 may include any number of organizations 102. Each of the organizations 102 is associated with an aspect of the field of medicine.

The organization₁ 102A is associated with a medical facility (e.g., a hospital) that provides a plurality of patients 104A-104N with medical care from a plurality of medical professionals 106, including a primary care physician 108, a primary surgeon 110, and a hospital pharmacist 112. These examples of the medical professionals 106 are merely exemplary to illustrate certain aspects of the concepts and technologies disclosed herein, and therefore should not be construed as being limiting in any way. In addition, the patients 104 may have one or more caregivers 114 such as in-home nurses, hospice care nurses, friends, and/or family members. The illustrated example shows a first caregiver (“caregiver₁”) 114A, a second caregiver (“caregivers”) 114B, and an n^{th} caregiver (“caregiverₙ”) 114N.

The organization₂ 102B is associated with a pharmacy chain that provides prescription medications to the plurality of patients 104A-104N. The organization₂ 102B is divided into locations, including pharmacies in a first city (“city₁”) 118A, pharmacies in a second city (“city₂”) 118B, and pharmacies in a third city (“city₃”) 118C.

The organization₃ 102C is associated with an emergency care facility that provides the plurality of patients 104A-104N with emergency medical care from a plurality of emergency room (“ER”) doctors 120, including a first ER doctor (“ER doctor₁”) 120A and second ER doctor (“ER doctors”) 120B, one or more imaging specialists 122 (e.g., radiography technicians, radiologists, etc.) of which only one imaging specialist 122 is shown, and one or more emergency pharmacies 124 of which only one emergency pharmacy 124 is shown.

Each of the organizations 102 contains one or more peer nodes 126. The organization¹ 102A associated with the medical facility includes a first peer node (“peer node₁”) 126A associated with the patients 104, a second peer node (“peer node₁”) 126B associated with the medical professionals 106, and a third peer node (“peer node₃”) 126C associated with the patients caregivers 114. The organizations 102B associated with the pharmacy chain includes a fourth peer node (“peer node₄”) 126D associated with the pharmacies in city₁ 118A, a fifth peer node (“peer node₅”) 126E associated with the pharmacies in city₂ 118B, and a sixth peer node (“peer node₆”) 126F associated with the pharmacies in city₃ 118C. The organization₃ 102C associated with the emergency care facility includes a seventh peer node (“peer node₇”) 126G associated with the emergency room doctors 120, an eight peer node (“peer node₈”) 126H associated with the imaging specialist 122, and a ninth peer node (“peer node₉”) 126I associated with the emergency pharmacy 124.

The peer nodes 126 can interact (e.g., communicate and transact) with one another via one or more channels 128. Each of the channels 128 provides a communication path among a specific set of the peer nodes 126 and applications (best shown in FIG. 1B) within the permissioned blockchain network 100. In accordance with the concepts and technologies disclosed herein, each of the channels 128 is associated with a different type of patient data 130 (best shown in FIG. 1C).

Turning briefly to FIG. 1C, each of the patients 104 can be associated with patient data 130. The patient data 130 can include personally identifiable data 132 that encompasses any data that may be used to identify the identity of the patient 104. For one reason or another, the patient 104 might not want their identity exposed to others, such as, for example, to the medical professionals 106, the emergency room doctors 120, the imaging specialist 122, and/or others. However, the patient 104 may want at least a portion of their health data 134, including medical history data 136, prescription (“Rx”) history data 138, and/or remote patient monitoring (“RPM”) device data 140, to be shared with others. The medical history data 136 can contain any data associated with the medical history of the patient 104, including any relevant information about the patient's past and present that may be relevant to his/her health. For example, the medical history data 136 can include any information about allergies, illnesses, surgeries, immunizations, exam results, test results, and the like. The prescription history data 138 can include any information about medicine prescribed to the patient 104. The RPM device data 140 can include data measured by one or more RPM devices 142 (best shown in FIG. 1B). Additional details in this regard will be described below.

Turning back to FIG. 1A, the peer nodes 126 that subscribe to a channel A 128A can share the patient medical history data 136. The peer nodes 126 that subscribe to a channel B 128B can share the Rx history data 138. The peer nodes 126 that subscribe to a channel C 128C can share the RPM device data 140. The patient₁ 104A is associated with and shares his/her associated patient medical history data 136 via the channel A 128A (shown as CH_A-1 128A-1). The patient₁ 104A also is associated with and shares his/her associated RX history data 138 via the channel B 128B (shown as CH_B-1 128B-1). The patient₁ 104A also is associated with and shares his/her associated RPM device data 140 via the channel C 128C (shown as CH_C-1 128C-1). The patient₂ 104B is associated with and shares his/her associated patient medical history data 136 via the channel A 128A (shown as CH_A-2 128A-2). The patient₂ 104B also is associated with and shares his/her associated RX history data 138 via the channel B 128B (shown as CH_B-2 128B-2). The patient₂ 104B also is associated with and shares his/her associated RPM device data 140 via the channel C 128C (shown as CH_C-2 128C-2). The patientₙ 104N is associated with and shares his/her associated patient medical history data 136 via the channel A 128A (shown as CH_A-N 128A-N). The patientₙ 104N also is associated with and shares his/her associated RX history data 138 via the channel B 128B (shown as CH_B-N 128B-N). The patientₙ 104B also is associated with and shares his/her associated RPM device data 140 via the channel C 128C (shown as CH_C-N 128C-N). The various users of each peer node—for example, the medical professionals 106 of the peer node₂ 126B, the caregivers 114 of the peer nodes, and so on can access one or more of the channels 128A-128C to which a specific peer node 126 is subscribed to obtain the patient medical history data 136, the RX history data 138, and the RPM device data 140, respectively. The users can be required to provide credentials to obtain such data. This concept is described in further detail with reference to FIG. 1B.

Each of the organizations 102 is associated with a membership service provider (“MSP”) 144 that identifies one or more certificate authorities (“CAs”) 146 that are trusted to define the members (i.e., the peer nodes 126 and users thereof) of the organization 102. The illustrated example is simplified with a single CA 146 for each of the peer nodes 126, but multiple CAs 146 per peer node 126 are contemplated. In some embodiments, the default CA is a PKI-based CA available as part of the Hyperledger® FABRIC. The CAs 146 each define rules for the users of the peer nodes 126 in the permissioned blockchain network 100. The CAs 146 can specify attributes to make one or more rules (e.g., only administrators may have access to a specific channel). The CAs 146 generate certificates for each of the users (e.g., the CA₂ 146B can generate certificates for each of the medical professionals 106). The process of generating certificates is known as enrollment, and the CA 146 issues the generated certificates to the users. Every operation performed on a ledger (best shown in FIG. 1B) is cryptographically signed with a certificate. For example, if one of the medical professionals 106 wants to query the ledger associated with the channel A 128A for at least a portion of the medical history data 136, this query must be signed by his/her certificate to ensure only authenticated users are allowed access to the ledger.

Turning now to FIG. 1B, additional details about the permissioned blockchain network 100 will be described, according to an illustrative embodiment. The illustrated embodiment shows details of the organization₁ 102A, including the peer nodes 126A-126C. Each of the peer nodes 126A-126C can host one or more instances of one or more chaincodes 148 (also known as “smart contracts”), and one or more instances of one or more distributed ledgers (“ledgers”) 150. The chaincodes 148 provide terms, data rules, concepts, definitions and processes to enable interactions and transactions to occur within the permissioned blockchain network 100. The ledgers 150 enable automatic enforcement of these terms, rules, etc. The ledgers 150 each contain immutable records of transactions generated by one or more of the chaincodes 148.

In some embodiments, the chaincode 148 can be a lifecycle system chaincode (“LSCC”) that runs in all of the peer nodes 126A-126C to handle package signing, install, instantiate, and upgrade chaincode requests. In some embodiments, the chaincode 148 can be a configuration system chaincode (“CSCC”) that runs in all of the peer nodes 126A-126C to handle changes to the configuration of the channel(s) 128, such as a policy update. In some embodiments, the chaincode 148 can be a query system chaincode (“QSCC”) that runs in all of the peer nodes 126A-126C to provide ledger application programming interfaces (“APIs”; not shown) which include block query, transaction query, and the like. As will be described in greater detail below, the peer nodes 126 may function, at least in part, as endorser peer nodes to endorse a proposed change to the ledger 150. As such, in some embodiments, the chaincode 148 can be an endorsement system chaincode (“ESCC”) that runs in endorser peer nodes to cryptographically sign a transaction response to provide an endorsement for a proposed change to the ledger 150. In some embodiments, the chaincode 148 can be a validation system chaincode (“VSCC”) that validates a transaction, including checking any endorsement policies and read-write set versioning.

Each of the peer nodes 126 is configured to execute and verify transactions with the ledger 150. The peer nodes 126 also can mark each transaction as valid or invalid, and can write or append transaction blocks 156 to the ledger 150. The Hyperledger® FABRIC software development kit (“SDK”) provides APIs that enable applications, such as one or more blockchain client applications described below, to connect to the peer node(s) 126, to invoke the chaincode(s) 148 to generate transactions, to submit the transactions to one or more orderer nodes 154 to be ordered and committed to the ledger(s) 150, and to receive events that signify completion of these processes.

One or more orderer nodes 154 can be part of the organization 102. In the illustrated example, each of the peer nodes 126A, 126B, 126C is associated with an orderer node 154A, 154B, 154C, respectively. Alternatively, the organization 102 may have a single orderer node 154 for multiple peer nodes 126, or multiple orderer nodes 154 for one or more of the peer nodes 126. As such, the illustrated example should not be construed as being limiting in any way. The orderer nodes 154A-154C provide services to order transactions, to create the transaction blocks 156, and to distribute the transaction blocks 156 to other peer nodes 126 that share the same ledger 150.

Each of the ledgers 150 is associated with one of the channels. Specifically, the channel A 128A is associated with a ledger A 150A, the channel B 128B is associated with a ledger B 150B, and the channel C 128C is associated with a ledger C 150C. The chaincode(s) 148 associated with each of the ledgers 150A-150C can be invoked by the peer nodes 126 to perform various operations.

The users, such as the patient 104, the medical professional 106, and the patient caregiver 114, are illustrated as being in association with devices. The patient 104 is shown as being in association with one or more of the RPM devices 142, the medical professional 106 is shown as being in association with one or more medical professional devices 158, and the patient caregiver 114 is shown as being in association with one or more caregiver devices 160. In addition, each of these devices is configured to execute a blockchain client application, shown as a patient blockchain application 162 to be executed by the RPM device(s) 142, a medical professional blockchain application 164 to be executed by the medical professional device(s) 158, and a caregiver blockchain application 166 to be executed by the caregiver device(s) 160.

The blockchain client applications 162, 164, 166 can connect to the peer nodes 126A, 126B, 126C, respectively. The blockchain client applications 162, 164, 166 can update the ledgers 150 associated with the channels 128 to which the peer nodes 126 are subscribed. Following the example introduced above with respect to FIG. 1A, the patient blockchain application 162 and the medical professional blockchain application 164 can update the ledgers A-C 150A-150C associated with the channels A-C 128A-128C, and the caregiver blockchain application 166 can update the ledger C 150C associated with the channel C 128C. The individual users, such as the primary care physician 108, the primary surgeon 110, and the hospital pharmacist 112, may have different access privileges based upon the consent of the patients 104. The example shown in FIG. 1A shows that the patient₁ 104A and the patient₂ 104B both have given consent to the primary care physician 108 for access to his/her medical history data 136 on the channel A 128A, the patient Rx history data 138 on the channel B 128B, and the RPM device data 140 on the channel C 128C. The primary surgeon 110 does not have access to the RPM device data 140 on the channel C 128C since, presumably, the primary surgeon 110 has no need for this information during surgery. The hospital pharmacist 112 only has access to the patient Rx history data 138 on the channel B 128B since the patient medical history 136 and the RPM device data 140 is likely outside his/her purview.

The blockchain client applications 162, 164, 166 can update one or more of the ledgers 150. Briefly, the blockchain client applications 162, 164, 166 rely on a group of peer nodes 126 referred to herein as endorsing peers to provide an endorsement for any proposed ledger update. The endorsements are collected as transactions and packaged into the transaction blocks 156 by the orderer nodes 154. The transaction blocks 156 are distributed back to the peer nodes 126 that share the same ledger 150, and each transaction block 156 is validated before the peer nodes 126 apply the proposed ledger update to the ledger 150. A more detailed example of this process will now be described.

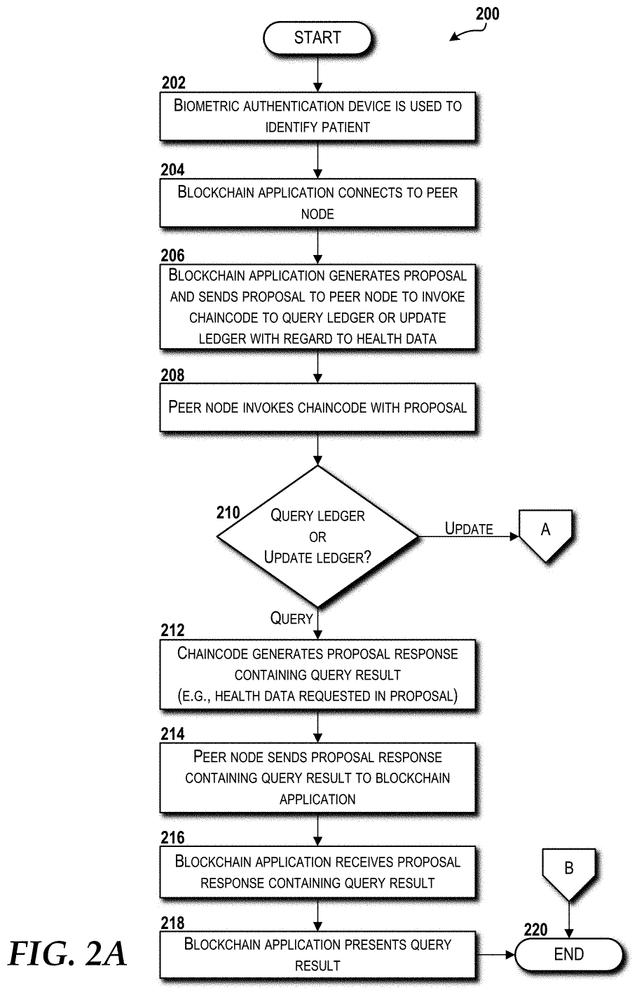

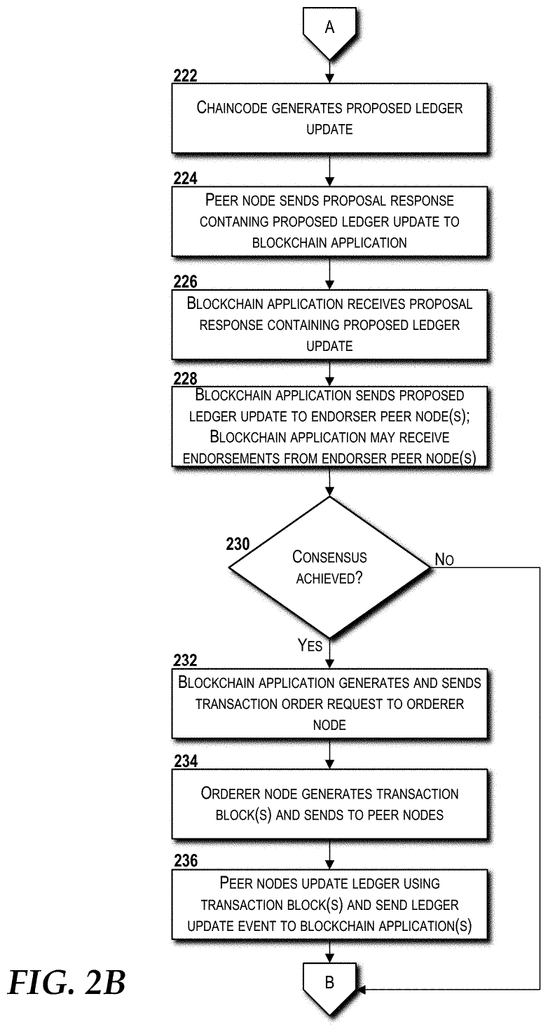

The medical professional device 158 executing the medical professional blockchain application 164 and interacting with the peer node₂ 126B and the orderer node₂ 154B will now be used as an example to explain how the ledger A 150A that contains the medical history data 136 of the patient 104 can be queried or updated. The medical professional blockchain application 164 connects (168) to the peer node₂ 126B, generates a proposal (170) to query or update the ledger A 150A, and sends the proposal (170) to the peer node₂ 126B to invoke the chaincode 148. The peer node₂ 126B invokes (172) the chaincode 148 to generate a proposal response (174) that contains a query result or a proposed ledger update depending on whether a query or an update was specified in the proposal (170). The medical professional blockchain application 164 receives and analyzes the proposal response (174). If the proposal (170) contained a query, such as one of the medical professionals 106 querying the ledger A 150A for the medical history data 136 of the patient 104 or some portion thereof, then the proposal response (174) can include the requested data. If the proposal (170) instead contained a proposed ledger update, such as one of the medical professionals 106 seeking to update the ledger A 150A to add to, remove, or modify the medical history data 136 of the patient 104 or some portion thereof, then the proposal response (174) can include a proposed ledger update that was generated by the chaincode 148. The peer node₂ 126B can send the proposed ledger update to other peer nodes 126 that have been identified as endorsers. If one or more of the endorser peer nodes fails to endorse the proposed ledger update, then the medical professional blockchain application 164 can abandon the ledger update. Additionally, the medical professional 106 may be alerted that he/she cannot, at this time, update the medical history data 136 of the patient. If, however, consensus is achieved among the endorser peer nodes (i.e., all endorser peer nodes have endorsed the proposed ledger update), then the medical professional blockchain application 164 can generate and send a transaction order request (176) to the orderer node₂ 154B. The transaction order request (176) can identify the ledger update that has been endorsed by the endorser peer nodes. The orderer node₂ 154B can generate the transaction blocks 156 and send the transaction blocks 156 to the peer node₂ 126B along with other peer nodes 126 that use the ledger A 150A. The peer node₂ 126B (and the other peer nodes 126 that receives the transaction blocks 156) can validate the transaction and update the ledger A 150A accordingly. After the ledger A 150A has been updated (178), the peer node₂ 126 can respond to the medical professional blockchain application 164 with a ledger update event (180) to indicate that the ledger A 150A has been updated successfully. The aforementioned query/update process is described in greater detail below with reference to FIGS. 2A-2B. Moreover, a similar process can occur between any of the blockchain client applications 162, 164, 166 and the peer nodes 126A, 126B, 126C to access one or more of the ledgers 150 accessible via the channels 128.

Access to the channels 128, and thereby, the ledgers 150, can be controlled based upon credentials of the user who is attempting to access the patient data 130. For example, the patient 104 can be associated with one or more patient credentials 182, the medical professional 106 can be associated with one or more medical professional credentials 184, and the patient caregiver 114 can be associated with one or more patient caregiver credentials 186. The credentials 182, 184, 186 can include access credentials such as username, password, personal identification number, any combination thereof, and/or the like. Additionally, aspects of the concepts and technologies disclosed herein provide additional security mechanisms to ensure that certain users, such as the medical professionals 106, are licensed to practice medicine or are otherwise licensed to care for the patient 104 in some capacity. For example, the medical professional credentials 184 for the primary care physician 108 (and/or other Doctors) may include information contained in a physical profile that can verify certain information about the medical professional 106, including medical school, graduate medical education, specialty board certifications, state licensure data and sanctions, Drug Enforcement Administration (“DEA”) registration, National Provider Identifier (“NPI”) number, combinations thereof, and/or the like. For other medical professionals 106, such as Emergency Medical Technicians (“EMTs”), Paramedics, and other medical professionals that are “in-the-field” for Ambulance, Fire, and/or other Emergency Services, the medical professional credentials 184 may include credentials such as a FIRSTNET subscriber identity module (“SIM”) identifier that can be used to provide, quick, trustworthy verification in-the-field.

Prior to receiving medical treatment or allowing access to the patient data 130, the patient 104 may be required to read and sign various forms, such as those relating to various aspects of the HIPPA Privacy Rule, other rules, and/or laws. Verbal consent is often sufficient, but the patient 104 may be incapacitated or otherwise unable to consent. Moreover, the patient 104 may not be able to provide any of their personally identifiable data 132 so that the medical professionals 106 and/or other users know the identity of the patient 104 to obtain at least a portion of the health data 134 of the patient 104. In accordance with the concepts and technologies disclosed herein, a biometric authentication device 188 may be used to obtain biometric data 190 from the patient 104 and use the biometric data 190 to obtain access to at least a portion of the patient data 130 associated with the patient 104 in accordance with the permissions established for the medical professionals 106 and other users described herein.

The biometric data 190 can uniquely identify the patient 104 from other patients (best shown in FIG. 1A) such as to identify the patient 104 by his/her unique biometric identity. The biometric authentication device 188 therefore can be or can include one or more biometric sensors. The biometric sensors can be standalone sensors or can be implemented in another computing system or device (not shown). In some embodiments, the biometric authentication device 188 is a smart phone, tablet, or similar mobile device that includes one or more biometric sensors. Dedicated medical devices that may be categorized as IoT devices are also contemplated.

The biometric authentication device 188 may use popular biometric sensors, such as, for example, fingerprint sensors, face recognition sensors, iris sensors, although the concepts and technologies disclosed herein should not be construed as being limiting to any particular type of biometric sensor, and therefore, also does not limit the type of biometric data 190 that can be obtained for use in accordance with the concepts and technologies disclosed herein. Moreover, the biometric authentication device 188 are intended to be representative of both hardware and software components used to obtain the biometric data 190. The software can be executed locally by the biometric authentication device 188 or via another system or device, such as the RPM device(s) 142, the medical professional device(s) 158, and/or the caregiver device(s) 160.

Turning now to FIGS. 2A-2B, a method 200 for querying or updating a ledger that contains patient data will be described, according to an exemplary embodiment. It should be understood that the operations of the methods disclosed herein are not necessarily presented in any particular order and that performance of some or all of the operations in an alternative order(s) is possible and is contemplated. The operations have been presented in the demonstrated order for ease of description and illustration. Operations may be added, omitted, and/or performed simultaneously, without departing from the scope of the concepts and technologies disclosed herein.

It also should be understood that the methods disclosed herein can be ended at any time and need not be performed in its entirety. Some or all operations of the methods, and/or substantially equivalent operations, can be performed by execution of computer-readable instructions included on a computer storage media, as defined herein. The term “computer-readable instructions,” and variants thereof, as used herein, is used expansively to include routines, applications, application modules, program modules, programs, components, data structures, algorithms, and the like. Computer-readable instructions can be implemented on various system configurations including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These states, operations, structural devices, acts, and modules may be implemented in software, in firmware, in special purpose digital logic, and any combination thereof. As used herein, the phrase “cause a processor to perform operations” and variants thereof is used to refer to causing one or more processors disclosed herein to perform operations.

For purposes of illustrating and describing some of the concepts of the present disclosure, the methods disclosed herein may be described as being performed, at least in part, by one of the processors via execution of one or more software modules. It should be understood that additional and/or alternative devices and/or network nodes can provide the functionality described herein via execution of one or more modules, applications, and/or other software. Thus, the illustrated embodiments are illustrative, and should not be viewed as being limiting in any way.

The method 200 will be described in context of the medical professional blockchain application 164 and its interactions with the peer node₂ 126B and the orderer₂ node 154B on behalf of the medical professional 106 to obtain at least a portion of the medical history data 136 that is stored in the ledger A 150A that securely maintains the medical history data 136 for the patient 104. It should be understood that the operations described herein as part of the method 200 are applicable to other scenarios, such as the caregiver blockchain application 166 and its interactions with the peer node₃ 126C and the orderer node₃ 154C on behalf of the patient caregiver 114 to obtain at least a portion of the RPM device data 140 that is stored in the ledger C 150C that securely maintains the RPM device data 140 for the patient 104.

The method 200 begins and proceeds to operation 202. At operation 202, the biometric authentication device 188 is used to identify the patient 104. For example, the medical professional 106 may use the biometric authentication device 188 to scan a fingerprint of the patient 104, scan an iris of one or both eyes of the patient 104, and/or scan the face of the patient 104 to obtain the biometric data 190 used to identify the patient 104. Those skilled in the art will appreciate the other types of biometric data 190 that can be used to identify the patient 104. The biometric authentication device 188 may be a standalone device, or may be integrated into another device such as the medical professional device 158.

From operation 202, the method 200 proceeds to operation 204. At operation 204, the medical professional blockchain application 164 connects to the peer node₂ 126B. In addition, at operation 204, the medical professional 106 may be prompted by the medical professional blockchain application 164 for the medical professional credentials 184 to verify that the medical professional 106 is authenticated to access at least a portion of the patient data 130 of the patient 104. Alternatively, another application executed by the medical professional device 158 and/or a remote authentication system (not shown) may be used, at least in part, to perform this authentication.

From operation 204, the method 200 proceeds to operation 206. At operation 206, the medical professional blockchain application 164 generates a proposal (170) and sends the proposal (170) to the peer node₂ 126B to invoke (172) the chaincode 148 to query the ledger A 150A or to update the ledger A 150A with regard to the patient data 130. From operation 206, the method 200 proceeds to operation 208. At operation 208, the peer node₂ 126B invokes the chaincode 148 with the proposal (170).

From operation 208, the method 200 proceeds to operation 210. At operation 210, the chaincode 138 determines if the proposal (170) is to query the ledger A 150A or update the ledger A 150A. If the chaincode 148 determines that the proposal (170) is to query the ledger A 150A, the method 200 proceeds to operation 212. At operation 212, the chaincode 148 generates a proposal response (174) containing a query result (e.g., the patient data 130 requested in the proposal (170)). From operation 212, the method 200 proceeds to operation 214. At operation 214, the peer node₂ 126B sends the proposal response (174) containing the query result to the medical professional blockchain application 164.

From operation 214, the method 200 proceeds to operation 216. At operation 216, the medical professional blockchain application 164 receives the proposal response (174). From operation 216, the method 200 proceeds to operation 218. At operation 218, the medical professional blockchain application 164 presents the query result. For example, the medical professional blockchain application 164 may present the query result to the medical professional 106.

From operation 218, the method 200 proceeds to operation 220. At operation 220, the method 200 can end.

Returning to operation 210, if the chaincode 148 determines that the proposal (170) is to update the ledger A 150A, the method 200 proceeds to operation 222 (shown in FIG. 2B). At operation 222, the chaincode 148 generates the proposed ledger update. From operation 222, the method 200 proceeds to operation 224. At operation 224, the peer node₂ 126B sends the proposal response (174) containing the proposed ledger update to the medical professional blockchain application 164. From operation 224, the method 200 proceeds to operation 226. At operation 226, the medical professional blockchain application 164 receives the proposal response (174) containing the proposed ledger update.

From operation 226, the method 200 proceeds to operation 228. At operation 228, the medical professional blockchain application 164 sends the proposed ledger update to a set of endorser peer nodes 126. Also at operation 228, the medical professional blockchain application 164 may receive endorsements from the endorser peer nodes 126. From operation 228, the method 200 proceeds to operation 230. At operation 230, the medical professional blockchain application 164 determines whether consensus has been achieved based upon whether endorsements have been received from all endorser peer nodes 126 in the set of endorser peer nodes 126. If, at operation 230, the medical professional blockchain application 164 determines that consensus has been achieved, the method 200 proceeds to operation 232. At operation 232, the medical professional blockchain application 164 generates and sends a transaction order request (176) to the orderer node₂ 154B. If, at operation 230, the medical professional blockchain application 164 determines that the consensus has not been achieved, the method 200 proceeds back to FIG. 2A, and in particular, operation 220, where the method 200 can end.

From operation 232, the method 200 proceeds to operation 234. At operation 234, the orderer node₂ 154B generates, based upon the transaction order request (176), the transaction blocks 156 to update (178) the ledger A 150A across all peer nodes 126 that have an instance of the ledger A 150A. From operation 234, the method 200 proceeds to operation 236. At operation 236, the peer nodes 126 that have an instance of the ledger A 150A update the ledger A 150A using the transaction blocks 156. Also at operation 236, the peer nodes 126 send a ledger update event (180) to the medical professional blockchain application 164. From operation 236, the method 200 returns to FIG. 1A, and particularly, operation 220. At operation 220, the method 200 can end.

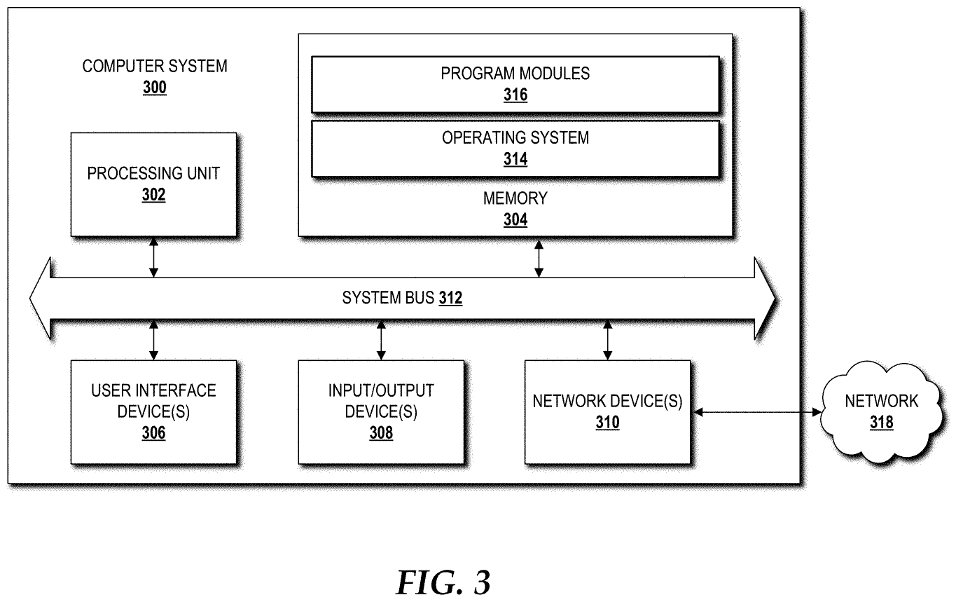

Turning now to FIG. 3 is a block diagram illustrating a computer system 300 configured to provide the functionality in accordance with various embodiments of the concepts and technologies disclosed herein. The systems, devices, and other components disclosed herein can utilize, at least in part, an architecture that is the same as or at least similar to the architecture of the computer system 300. For example, the peer nodes 126, the RPM device(s) 142, the medical professional device(s), the orderer nodes 154, the caregiver device(s) 160, the biometric authentication device 188, and/or other devices, systems, and nodes described herein can utilize, at least in part, an architecture that is the same as or at least similar to the architecture of the computer system 300. It should be understood, however, that modification to the architecture may be made to facilitate certain interactions among elements described herein.

The computer system 300 includes a processing unit 302, a memory 304, one or more user interface devices 306, one or more I/O devices 308, and one or more network devices 310, each of which is operatively connected to a system bus 312. The bus 312 enables bi-directional communication between the processing unit 302, the memory 304, the user interface devices 306, the I/O devices 308, and the network devices 310.

The processing unit 302 may be a standard central processor that performs arithmetic and logical operations, a more specific purpose programmable logic controller (“PLC”), a programmable gate array, or other type of processor known to those skilled in the art and suitable for controlling the operation of the server computer. Processing units are generally known, and therefore are not described in further detail herein.

The memory 304 communicates with the processing unit 302 via the system bus 312. In some embodiments, the memory 304 is operatively connected to a memory controller (not shown) that enables communication with the processing unit 302 via the system bus 312. The illustrated memory 304 includes an operating system 314 and one or more program modules 316. The operating system 314 can include, but is not limited to, members of the WINDOWS, WINDOWS CE, and/or WINDOWS MOBILE families of operating systems from MICROSOFT CORPORATION, the LINUX family of operating systems, the SYMBIAN family of operating systems from SYMBIAN LIMITED, the BREW family of operating systems from QUALCOMM CORPORATION, the MAC OS, OS X, and/or iOS families of operating systems from APPLE CORPORATION, the FREEBSD family of operating systems, the SOLARIS family of operating systems from ORACLE CORPORATION, other operating systems, and the like.

The program modules 316 may include various software and/or program modules to perform the various operations described herein. The program modules 316 and/or other programs can be embodied in computer-readable media containing instructions that, when executed by the processing unit 302, perform various operations such as those described herein. According to embodiments, the program modules 316 may be embodied in hardware, software, firmware, or any combination thereof.

By way of example, and not limitation, computer-readable media may include any available computer storage media or communication media that can be accessed by the computer system 300. Communication media includes computer-readable instructions, data structures, program modules, or other data in a modulated data signal such as a carrier wave or other transport mechanism and includes any delivery media. The term “modulated data signal” means a signal that has one or more of its characteristics changed or set in a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Computer storage media includes volatile and non-volatile, removable and non-removable media implemented in any method or technology for storage of information such as computer-readable instructions, data structures, program modules, or other data. Computer storage media includes, but is not limited to, RAM, ROM, Erasable Programmable ROM (“EPROM”), Electrically Erasable Programmable ROM (“EEPROM”), flash memory or other solid state memory technology, CD-ROM, digital versatile disks (“DVD”), or other optical storage, magnetic cassettes, magnetic tape, magnetic disk storage or other magnetic storage devices, or any other medium which can be used to store the desired information and which can be accessed by the computer system 300. In the claims, the phrase “computer storage medium” and variations thereof does not include waves or signals per se and/or communication media.

The user interface devices 306 may include one or more devices with which a user accesses the computer system 300. The user interface devices 306 may include, but are not limited to, computers, servers, personal digital assistant (“PDAs”), cellular phones, or any suitable computing devices. The I/O devices 308 enable a user to interface with the program modules 316. In one embodiment, the I/O devices 308 are operatively connected to an I/O controller (not shown) that enables communication with the processing unit 302 via the system bus 312. The I/O devices 308 may include one or more input devices, such as, but not limited to, a keyboard, a mouse, or an electronic stylus. Further, the I/O devices 308 may include one or more output devices, such as, but not limited to, a display screen or a printer. In some embodiments, the I/O devices 308 can be used for manual controls for operations to exercise under certain emergency situations.

The network devices 310 enable the computer system 300 to communicate with other networks or remote systems via a network 318. Examples of the network devices 310 include, but are not limited to, a modem, a radio frequency (“RF”) or infrared (“IR”) transceiver, a telephonic interface, a bridge, a router, or a network card. The network 318 may be or may include a wireless network such as, but not limited to, a Wireless Local Area Network (“WLAN”), a Wireless Wide Area Network (“WWAN”), a Wireless Personal Area Network (“WPAN”) such as provided via BLUETOOTH technology, a Wireless Metropolitan Area Network (“WMAN”) such as a WiMAX network or metropolitan cellular network. Alternatively, the network 318 may be or may include a wired network such as, but not limited to, a Wide Area Network (“WAN”), a wired Personal Area Network (“PAN”), or a wired Metropolitan Area Network (“MAN”).

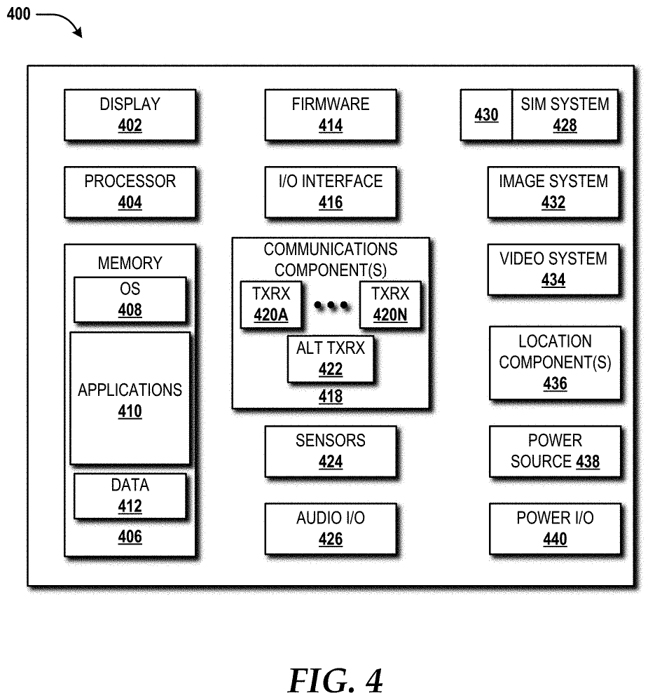

Turning now to FIG. 4, an illustrative mobile device 400 and components thereof will be described. In some embodiments, the peer nodes 126, the RPM device(s) 142, the medical professional device(s), the orderer nodes 154, the caregiver device(s) 160, the biometric authentication device 188, and/or other devices, systems, and nodes described herein can be configured like the mobile device 400. While connections are not shown between the various components illustrated in FIG. 4, it should be understood that some, none, or all of the components illustrated in FIG. 4 can be configured to interact with one other to carry out various device functions. In some embodiments, the components are arranged so as to communicate via one or more busses (not shown). Thus, it should be understood that FIG. 4 and the following description are intended to provide a general understanding of a suitable environment in which various aspects of embodiments can be implemented, and should not be construed as being limiting in any way.

As illustrated in FIG. 4, the mobile device 400 can include a display 402 for displaying data. According to various embodiments, the display 402 can be configured to display various graphical user interface (“GUI”) elements, text, images, video, virtual keypads and/or keyboards, messaging data, notification messages, metadata, internet content, device status, time, date, calendar data, device preferences, map and location data, combinations thereof, and/or the like. The mobile device 400 also can include a processor 404 and a memory or other data storage device (“memory”) 406. The processor 404 can be configured to process data and/or can execute computer-executable instructions stored in the memory 406. The computer-executable instructions executed by the processor 404 can include, for example, an operating system 408, one or more applications 410, other computer-executable instructions (e.g., associated with the software 120) stored in the memory 406, or the like. In some embodiments, the applications 410 also can include a user interface (“UP”) application (not illustrated in FIG. 4).

The UI application can interface with the operating system 408 to facilitate user interaction with functionality and/or data stored at the mobile device 400 and/or stored elsewhere. In some embodiments, the operating system 408 can include a member of the SYMBIAN OS family of operating systems from SYMBIAN LIMITED, a member of the WINDOWS MOBILE OS and/or WINDOWS PHONE OS families of operating systems from MICROSOFT CORPORATION, a member of the PALM WEBOS family of operating systems from HEWLETT PACKARD CORPORATION, a member of the BLACKBERRY OS family of operating systems from RESEARCH IN MOTION LIMITED, a member of the IOS family of operating systems from APPLE INC., a member of the ANDROID OS family of operating systems from GOOGLE INC., and/or other operating systems. These operating systems are merely illustrative of some contemplated operating systems that may be used in accordance with various embodiments of the concepts and technologies described herein and therefore should not be construed as being limiting in any way.

The UI application can be executed by the processor 404 to aid a user entering content, viewing account information, answering/initiating calls, entering/deleting data, entering and setting user IDs and passwords for device access, configuring settings, manipulating address book content and/or settings, multimode interaction, interacting with other applications 410, and otherwise facilitating user interaction with the operating system 408, the applications 410, and/or other types or instances of data 412 that can be stored at the mobile device 400. According to various embodiments, the applications 410 can include, for example, any of the blockchain client applications 162, 164, 166, presence applications, visual voice mail applications, messaging applications, text-to-speech and speech-to-text applications, add-ons, plug-ins, email applications, music applications, video applications, camera applications, location-based service applications, power conservation applications, game applications, productivity applications, entertainment applications, enterprise applications, combinations thereof, and the like.

The applications 410, the data 412, and/or portions thereof can be stored in the memory 406 and/or in a firmware 414, and can be executed by the processor 404. The firmware 414 also can store code for execution during device power up and power down operations. It can be appreciated that the firmware 414 can be stored in a volatile or non-volatile data storage device including, but not limited to, the memory 406 and/or a portion thereof.

The mobile device 400 also can include an input/output (“I/O”) interface 416. The I/O interface 416 can be configured to support the input/output of data such as database data, location information, user information, organization information, presence status information, user IDs, passwords, and application initiation (start-up) requests. In some embodiments, the I/O interface 416 can include a hardwire connection such as universal serial bus (“USB”) port, a mini-USB port, a micro-USB port, an audio jack, a PS2 port, an Institute of Electrical and Electronics Engineers (“IEEE”) 1394 (“FIREWIRE”) port, a serial port, a parallel port, an Ethernet (RJ45) port, an RJ10 port, a proprietary port, combinations thereof, or the like. In some embodiments, the mobile device 400 can be configured to synchronize with another device to transfer content to and/or from the mobile device 400. In some embodiments, the mobile device 400 can be configured to receive updates to one or more of the applications 410 via the I/O interface 416, though this is not necessarily the case. In some embodiments, the I/O interface 416 accepts I/O devices such as keyboards, keypads, mice, interface tethers, printers, plotters, external storage, touch/multi-touch screens, touch pads, trackballs, joysticks, microphones, remote control devices, displays, projectors, medical equipment (e.g., stethoscopes, heart monitors, and other health metric monitors), modems, routers, external power sources, docking stations, combinations thereof, and the like. It should be appreciated that the I/O interface 416 may be used for communications between the mobile device 400 and a network device or local device.

The mobile device 400 also can include a communications component 418. The communications component 418 can be configured to interface with the processor 404 to facilitate wired and/or wireless communications with one or more networks such as one or more IP access networks and/or one or more circuit access networks. In some embodiments, other networks include networks that utilize non-cellular wireless technologies such as WI-FI or WIMAX. In some embodiments, the communications component 418 includes a multimode communications subsystem for facilitating communications via the cellular network and one or more other networks.

The communications component 418, in some embodiments, includes one or more transceivers. The one or more transceivers, if included, can be configured to communicate over the same and/or different wireless technology standards with respect to one another. For example, in some embodiments one or more of the transceivers of the communications component 418 may be configured to communicate using Global System for Mobile communications (“GSM”), Code-Division Multiple Access (“CDMA”) ONE, CDMA2000, Long-Term Evolution (“LTE”), and various other 2G, 2.5G, 3G, 4G, 5G, and greater generation technology standards. Moreover, the communications component 418 may facilitate communications over various channel access methods (which may or may not be used by the aforementioned standards) including, but not limited to, Time-Division Multiple Access (“TDMA”), Frequency-Division Multiple Access (“FDMA”), Wideband CDMA (“W-CDMA”), Orthogonal Frequency-Division Multiplexing (“OFDM”), Space-Division Multiple Access (“SDMA”), and the like.

In addition, the communications component 418 may facilitate data communications using Generic Packet Radio Service (“GPRS”), Enhanced Data Rates for Global Evolution (“EDGE”), the High-Speed Packet Access (“HSPA”) protocol family including High-Speed Download Packet Access (“HSDPA”), Enhanced Uplink (“EUL”) or otherwise termed High-Speed Upload Packet Access (“HSUPA”), HSPA+, and various other current and future wireless data access standards. In the illustrated embodiment, the communications component 418 can include a first transceiver (“TxRx”) 420A that can operate in a first communications mode (e.g., GSM). The communications component 418 also can include an N^{th} transceiver (“TxRx”) 420N that can operate in a second communications mode relative to the first transceiver 420A (e.g., UMTS). While two transceivers 420A-420N (hereinafter collectively and/or generically referred to as “transceivers 420”) are shown in FIG. 4, it should be appreciated that less than two, two, and/or more than two transceivers 420 can be included in the communications component 418.

The communications component 418 also can include an alternative transceiver (“Alt TxRx”) 422 for supporting other types and/or standards of communications. According to various contemplated embodiments, the alternative transceiver 422 can communicate using various communications technologies such as, for example, WI-FI, WIMAX, BLUETOOTH, infrared, infrared data association (“IRDA”), near-field communications (“NFC”), ZIGBEE, other radio frequency (“RF”) technologies, combinations thereof, and the like.

In some embodiments, the communications component 418 also can facilitate reception from terrestrial radio networks, digital satellite radio networks, internet-based radio service networks, combinations thereof, and the like. The communications component 418 can process data from a network such as the Internet, an intranet, a broadband network, a WI-FI hotspot, an Internet service provider (“ISP”), a digital subscriber line (“DSL”) provider, a broadband provider, combinations thereof, or the like.

The mobile device 400 also can include one or more sensors 424. The sensors 424 can include biometric sensors (as part of or in lieu of the biometric authentication device 188), temperature sensors, light sensors, air quality sensors, movement sensors, orientation sensors, noise sensors, proximity sensors, or the like. As such, it should be understood that the sensors 424 can include, but are not limited to, accelerometers, magnetometers, gyroscopes, infrared sensors, noise sensors, microphones, combinations thereof, or the like. Additionally, audio capabilities for the mobile device 400 may be provided by an audio I/O component 426. The audio I/O component 426 of the mobile device 400 can include one or more speakers for the output of audio signals, one or more microphones for the collection and/or input of audio signals, and/or other audio input and/or output devices.

The illustrated mobile device 400 also can include a subscriber identity module (“SIM”) system 428. The SIM system 428 can include a universal SIM (“USIM”), a universal integrated circuit card (“UICC”) and/or other identity devices. The SIM system 428 can include and/or can be connected to or inserted into an interface such as a slot interface 430. In some embodiments, the slot interface 430 can be configured to accept insertion of other identity cards or modules for accessing various types of networks. Additionally, or alternatively, the slot interface 430 can be configured to accept multiple subscriber identity cards. Because other devices and/or modules for identifying users and/or the mobile device 400 are contemplated, it should be understood that these embodiments are illustrative, and should not be construed as being limiting in any way.

The mobile device 400 also can include an image capture and processing system 432 (“image system”). The image system 432 can be configured to capture or otherwise obtain photos, videos, and/or other visual information. As such, the image system 432 can include cameras, lenses, charge-coupled devices (“CCDs”), combinations thereof, or the like. The mobile device 400 may also include a video system 434. The video system 434 can be configured to capture, process, record, modify, and/or store video content. Photos and videos obtained using the image system 432 and the video system 434, respectively, may be added as message content to a multimedia message service (“MMS”) message, email message, and sent to another mobile device. The video and/or photo content also can be shared with other devices via various types of data transfers via wired and/or wireless communication devices as described herein.

The mobile device 400 also can include one or more location components 436. The location components 436 can be configured to send and/or receive signals to determine a geographic location of the mobile device 400. According to various embodiments, the location components 436 can send and/or receive signals from global positioning system (“GPS”) devices, assisted GPS (“A-GPS”) devices, WI-FI/WIMAX and/or cellular network triangulation data, combinations thereof, and the like. The location component 436 also can be configured to communicate with the communications component 418 to retrieve triangulation data for determining a location of the mobile device 400. In some embodiments, the location component 436 can interface with cellular network nodes, telephone lines, satellites, location transmitters and/or beacons, wireless network transmitters and receivers, combinations thereof, and the like. In some embodiments, the location component 436 can include and/or can communicate with one or more of the sensors 424 such as a compass, an accelerometer, and/or a gyroscope to determine the orientation of the mobile device 400. Using the location component 436, the mobile device 400 can generate and/or receive data to identify its geographic location, or to transmit data used by other devices to determine the location of the mobile device 400. The location component 436 may include multiple components for determining the location and/or orientation of the mobile device 400.

The illustrated mobile device 400 also can include a power source 438. The power source 438 can include one or more batteries, power supplies, power cells, and/or other power subsystems including alternating current (“AC”) and/or direct current (“DC”) power devices. The power source 438 also can interface with an external power system or charging equipment via a power I/O component 440. Because the mobile device 400 can include additional and/or alternative components, the above embodiment should be understood as being illustrative of one possible operating environment for various embodiments of the concepts and technologies described herein. The described embodiment of the mobile device 400 is illustrative, and should not be construed as being limiting in any way.

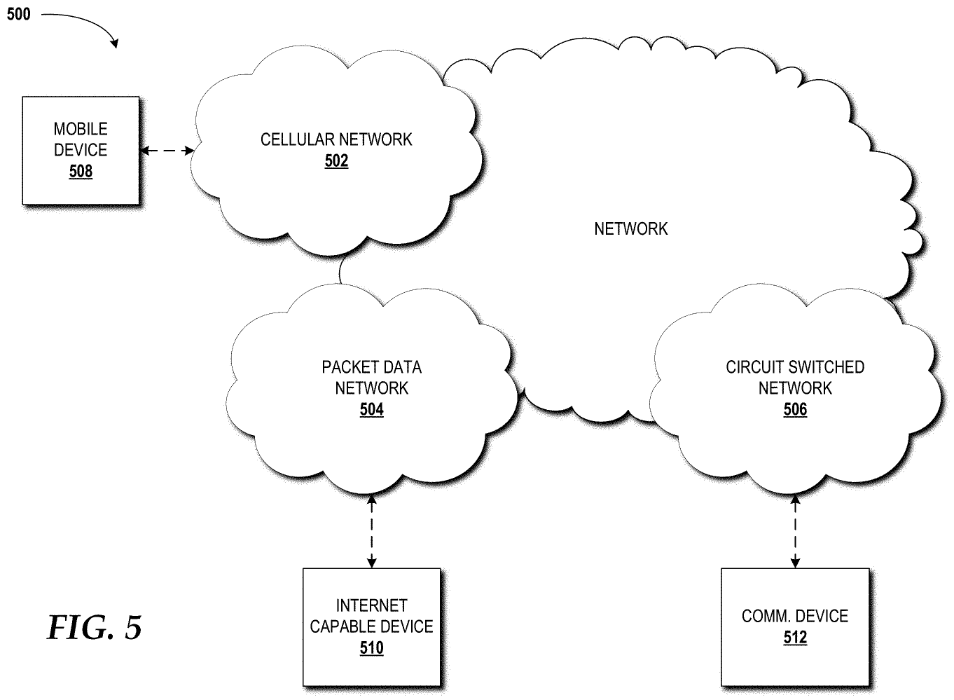

Turning now to FIG. 5, details of a network 500 are illustrated, according to an illustrative embodiment. The network 318 illustrated in FIG. 3 can be configured like the network 500. Moreover, the various devices and nodes described herein can communicate over one or more networks that can be configured like the network 500. The network 500 includes a cellular network 502, a packet data network 504, and a circuit switched network 506, for example, a publicly switched telephone network (“PSTN”).

The cellular network 502 includes various components such as, but not limited to, base transceiver stations (“BTSs”), nodeBs (“NBs”), eNBs, base station controllers (“BSCs”), radio network controllers (“RNCs”), mobile switching centers (“MSCs”), MMES, SGWs, PGWs, short message service centers (“SMSCs”), multimedia messaging service centers (“MMSCs”), home location registers (“HLRs”), home subscriber servers (“HS Ss”), visitor location registers (“VLRs”), charging platforms, billing platforms, voicemail platforms, GPRS core network components, location service nodes, an IP Multimedia Subsystem (“IMS”), and the like. The cellular network 502 also includes radios and nodes for receiving and transmitting voice, data, and combinations thereof to and from radio transceivers, networks, the packet data network 504, and the circuit switched network 506.

A mobile communications device 508, such as, for example, the device 108, a computing device, a cellular telephone, a mobile terminal, a PDA, a laptop computer, a handheld computer, and combinations thereof, can be operatively connected to the cellular network 502. The cellular network 502 can be configured as a 2G GSM network and can provide data communications via GPRS and/or EDGE. Additionally, or alternatively, the cellular network 502 can be configured as a 3G UMTS network and can provide data communications via the HSPA protocol family, for example, HSDPA, EUL (also referred to as HSUPA), and HSPA+. The cellular network 502 also is compatible with 4G mobile communications standards as well as evolved and future mobile standards.

The packet data network 504 includes various systems, devices, and/or nodes, for example, the peer nodes 126, the RPM device(s) 142, the medical professional device(s), the orderer nodes 154, the caregiver device(s) 160, the biometric authentication device 188, servers, computers, databases, and other devices in communication with another, as is generally known. The packet data network 504 devices are accessible via one or more network links. The servers often store various files that are provided to a requesting device such as, for example, a computer, a terminal, a smartphone, or the like. Typically, the requesting device includes software (a “browser”) for executing a web page in a format readable by the browser or other software. Other files and/or data may be accessible via “links” in the retrieved files, as is generally known. In some embodiments, the packet data network 504 includes or is in communication with the Internet. The circuit switched network 506 includes various hardware and software for providing circuit switched communications. The circuit switched network 506 may include, or may be, what is often referred to as a plain old telephone system (“POTS”). The functionality of a circuit switched network 506 or other circuit-switched network are generally known and will not be described herein in detail.

The illustrated cellular network 502 is shown in communication with the packet data network 504 and the circuit switched network 506, though it should be appreciated that this is not necessarily the case. One or more Internet-capable devices 510, for example, the peer nodes 126, the RPM device(s) 142, the medical professional device(s), the orderer nodes 154, the caregiver device(s) 160, the biometric authentication device 188, a PC, a laptop, a portable device, or another suitable device, can communicate with one or more cellular networks 502, and devices connected thereto, through the packet data network 504. It also should be appreciated that the Internet-capable device 510 can communicate with the packet data network 504 through the circuit switched network 506, the cellular network 502, and/or via other networks (not illustrated).

As illustrated, a communications device 512, for example, a telephone, facsimile machine, modem, computer, or the like, can be in communication with the circuit switched network 506, and therethrough to the packet data network 504 and/or the cellular network 502. It should be appreciated that the communications device 512 can be an Internet-capable device, and can be substantially similar to the Internet-capable device 510. In the specification, the network 500 is used to refer broadly to any combination of the networks 502, 504, 506. It should be appreciated that substantially all of the functionality described with reference to the network 500 can be performed by the cellular network 502, the packet data network 504, and/or the circuit switched network 506, alone or in combination with other networks, network elements, and the like.

Based on the foregoing, it should be appreciated that concepts and technologies directed to biometric authentication for access to medical information on a distributed ledger have been disclosed herein. Although the subject matter presented herein has been described in language specific to computer structural features, methodological and transformative acts, specific computing machinery, and computer-readable media, it is to be understood that the concepts and technologies disclosed herein are not necessarily limited to the specific features, acts, or media described herein. Rather, the specific features, acts and mediums are disclosed as example forms of implementing the concepts and technologies disclosed herein.

The subject matter described above is provided by way of illustration only and should not be construed as limiting. Various modifications and changes may be made to the subject matter described herein without following the example embodiments and applications illustrated and described, and without departing from the true spirit and scope of the embodiments of the concepts and technologies disclosed herein.

1. A method comprising:
receiving, by a device comprising a processor, biometric data identifying a patient, wherein a biometric authentication device identifies the patient and provides the biometric data;
executing, by the device, a blockchain application to connect the device to a peer node, wherein the peer node hosts a chaincode and a ledger, and wherein the ledger stores health data associated with the patient;
generating, by the device, a proposal directed to the peer node, wherein the proposal is to invoke the chaincode to interact with the ledger with regard to the health data;
sending, by the device, the proposal to the peer node; and
receiving, by the device, a proposal response from the peer node.

2. The method of claim 1, wherein the biometric authentication device is a standalone device.

3. The method of claim 1, wherein the biometric authentication device is part of the device executing the blockchain application.

4. The method of claim 1, wherein the ledger is associated with a specific channel of a plurality of channels associated with the health data; and wherein a plurality of peer nodes comprising the peer node, and the blockchain application are associated with the specific channel.

5. The method of claim 4, wherein the ledger comprises:
medical history data associated with the patient;
patient prescription history data associated with the patient; or
remote patient monitoring device data associated with the patient.

6. The method of claim 5, wherein the proposal is to query the ledger for at least a portion of the health data stored by the ledger; and wherein the proposal response comprises a query result comprising at least the portion of the health data.

7. The method of claim 5, wherein the proposal is to update the ledger; and further comprising:
receiving, by the device, the proposal response comprising a proposed ledger update;
sending, by the device, the proposed ledger update to a plurality of endorser peer nodes;
receiving, by the device, from the plurality of endorser peer nodes, endorsements of the proposed ledger update such that consensus among the plurality of endorser peer nodes is achieved;
generating and sending, by the device, a transaction order request to an orderer node, wherein the orderer node generates a transaction block based upon the transaction order request to update the ledger in accordance with the proposed ledger update; and
receiving, by the device, a ledger update event to indicate that the ledger has been updated by the peer node in accordance with the proposed ledger update.

8. A permissioned blockchain network comprising:
an organization comprising a plurality of peer nodes and an orderer node;
a first peer node of the plurality of peer nodes associated with a first group of users, wherein the first group of users comprises a plurality of patients;
a second peer node of the plurality of peer nodes associated with a second group of users, wherein the second group of users comprises a plurality of medical professionals;
a third peer node of the plurality of peer nodes associated with a third group of users, wherein the third group of users comprises a plurality of caregivers; and
a plurality of channels through which the plurality of peer nodes communicate to update a plurality of ledgers, wherein one ledger of the plurality of ledgers is associated with one channel of the plurality of channels, wherein a first ledger of the plurality of ledgers comprises medical history data associated with the plurality of patients, wherein a second ledger of the plurality of ledgers comprises prescription history data associated with the plurality of patients, and wherein a third ledger of plurality of ledgers comprises remote patient monitoring device data associated with the plurality of patients.

9. The permissioned blockchain network of claim 8, further comprising:
a device comprising a processor executing a blockchain application to perform operations comprising
connecting the device to a specific peer node of the plurality of peer nodes;
generating a proposal directed to the specific peer node, wherein the proposal is to invoke a specific chaincode to interact with a specific ledger;
sending the proposal to the specific peer node; and
receiving a proposal response from the specific peer node.

10. The permissioned blockchain network of claim 9, further comprising:
a biometric authentication device comprising a further processor executing instructions to perform operations comprising identifying a specific patient of the plurality of patients via biometric data obtained from a biometric sensor of the biometric authentication device.

11. The permissioned blockchain network of claim 10, wherein the biometric authentication device is part of the device executing the blockchain application.

12. The permissioned blockchain network of claim 10, wherein the proposal is to query the specific ledger for at least a portion of health data stored by the specific ledger, wherein the health data comprises the medical history data associated with the specific patient, the prescription history data associated with the specific patient, or the remote patient monitoring device data associated with the specific patient; and wherein the proposal response comprises a query result comprising at least the portion of the health data.

13. The permissioned blockchain network of claim 10, wherein the proposal is to update the specific ledger; and wherein the device executes the blockchain application to perform operations further comprising:
receiving the proposal response comprising a proposed ledger update;
sending proposed ledger update to a plurality of endorser peer nodes;
receiving, from the plurality of endorser peer nodes, endorsements of the proposed ledger update such that consensus among the plurality of endorser peer nodes is achieved;
generating and sending a transaction order request to the orderer node, wherein the orderer node generates a transaction block based upon the transaction order request to update the specific ledger in accordance with the proposed ledger update; and
receiving a ledger update event to indicate that the specific ledger has been updated by the specific peer node in accordance with the proposed ledger update.

14. A computer-readable storage medium comprising computer-executable instructions of a blockchain application that, when executed by a processor, cause the processor to perform operations comprising:
connecting to a peer node, wherein the peer node hosts a chaincode and a ledger, and wherein the ledger stores health data associated with a patient;
generating a proposal directed to the peer node, wherein the proposal is to invoke the chaincode to interact with the ledger with regard to the health data;
sending the proposal to the peer node; and
receiving a proposal response from the peer node.

15. The computer-readable storage medium of claim 14, wherein the operations further comprise identifying the patient based upon biometric data obtained by a biometric sensor.

16. The computer-readable storage medium of claim 14, wherein the ledger is associated with a specific channel of a plurality of channels associated with the health data; and wherein a plurality of peer nodes comprising the peer node, and the blockchain application are associated with the specific channel.

17. The computer-readable storage medium of claim 16, wherein the ledger comprises:
medical history data associated with the patient;
patient prescription history data associated with the patient; or
remote patient monitoring device data associated with the patient.

18. The computer-readable storage medium of claim 17, wherein the proposal is to query the ledger for at least a portion of the health data stored by the ledger; and wherein the proposal response comprises a query result comprising at least the portion of the health data.

19. The computer-readable storage medium of claim 17, wherein the proposal is to update the ledger; and wherein the operations further comprise:
receiving the proposal response comprising a proposed ledger update;
sending the proposed ledger update to a plurality of endorser peer nodes;
receiving, from the plurality of endorser peer nodes endorsements of the proposed ledger update such that consensus among the plurality of endorser peer nodes is achieved;
generating and sending a transaction order request to an orderer node, wherein the orderer node generates a transaction block based upon the transaction order request to update the ledger in accordance with the proposed ledger update; and
receiving a ledger update event to indicate that the ledger has been updated by the peer node in accordance with the proposed ledger update.

20. The computer-readable storage medium of claim 19, wherein the proposed ledger update comprises adding, removing, or modifying at least a portion of the ledger based upon input received by a medical professional.
